(19) United States
(12) Patent Application Publication
(10) Pub. No.: US 2025/0060432 A1
(43) Pub. Date: Feb. 20, 2025

(54) ASCERTAINING A PERFORMANCE VALUE OF AN MRT SYSTEM

(71) Applicant: Siemens Healthineers AG, Forchheim (DE)

(72) Inventor: Volker Schnetter, Nürnberg (DE)

(21) Appl. No.: 18/796,548

(22) Filed: Aug. 7, 2024

(30) Foreign Application Priority Data
Aug. 16, 2023 (EP) 23191605.7

(51) Int. Cl. G01R 33/28; G01R 33/54; G01R 33/58

(52) U.S. Cl. CPC G01R 33/288; G01R 33/543; G01R 33/58

(57) ABSTRACT

Systems and methods for ascertaining a performance value of an MRT system including where a reference frequency distribution of reference deposition values of examined reference objects is received. At least one examination deposition value for an MRT examination of at least one examination object is received by the MRT system. The performance value of the MRT system is ascertained using the reference frequency distribution and the at least one examination deposition value.

### CROSS REFERENCE TO RELATED APPLICATIONS

This application claims the benefit EP 23191605.7 filed on Aug. 16, 2023, which is hereby incorporated by reference in its entirety.

### FIELD

Embodiments relates to a method for ascertaining a performance value of an MRT system.

### BACKGROUND

In medical technology, magnetic resonance tomography (MRT; or magnetic resonance imaging, MRI) is characterized by high soft tissue contrasts. Typically, a patient is positioned in a main magnetic field of an MRT system. During an MRT examination, radio frequency (RF) pulses may be radiated into the patient using a radio frequency antenna unit of a magnetic resonance apparatus. The generated RF pulses excite nuclear spins in the patient, triggering location-encoded magnetic resonance signals. The magnetic resonance signals are received by the MRT system and used to reconstruct magnetic resonance images.

For the operator of an MRT system, the highest possible performance is desirable. The performance of an MRT system may be expressed, for example, in the speed of the MRT examination and/or a number of measured slices and/or a resulting image quality.

### BRIEF SUMMARY AND DESCRIPTION

The scope of the embodiments is defined solely by the appended claims and is not affected to any degree by the statements within this summary. The present embodiments may obviate one or more of the drawbacks or limitations in the related art. Independent of the grammatical term usage, individuals with male, female or other gender identities are included within the term.

Embodiments ascertain a performance value of an MRT system, for example in order to be able to reliably recognize and evaluate possible changes in the performance.

A computer-implemented method for ascertaining a performance value of an MRT system is provided. A reference frequency distribution of reference deposition values of examined reference objects is received. Furthermore, at least one examination deposition value for an MRT examination of at least one examination object is received, for example ascertained, by the MRT system. The performance value of the MRT system is ascertained using the reference frequency distribution and the at least one examination deposition value.

Ascertaining the performance value using the reference frequency distribution allows the performance of the MRT system to be evaluated statistically. This means that the performance of the MRT system may be assessed particularly reliably. A performance value of an MRT system may be a measure of the performance and/or functionality of the MRT system.

The proposed method takes patient variation into account when assessing the performance of the MRT system. Systematic deviations may be better distinguished from random deviations.

The deposition values are values that are regularly ascertained anyway for performing MRT examinations, so that they are already available as reference deposition values or are ascertained anyway as examination deposition values, i.e. are not only ascertained for the purpose of ascertaining the performance value.

The reference objects may be patients who have been examined with one or more reference MRT systems. The examination objects may be patients who are examined with the MRT system for which the performance value is to be ascertained.

The reference frequency distribution may be generated for example from existing MRT examination data sets. For example, the frequency distribution of the deposition values of the examined reference objects is ascertained. The frequency distribution describes for example a respective number of the respective reference deposition values, that were ascertained for the objects examined in a previous MRT examination with the at least one object parameter value. The reference deposition values relate to the examined reference objects, that for example have an object parameter value as the examination parameter value. The object parameter value may, for example, relate to a specific value of a mass of the examined object or a specific range of values of the masses of the examined objects. In other words, the reference frequency distribution refers to the objects already examined that have the at least one object parameter value as an examination parameter value. The frequency distribution may, for example, describe which deposition values were determined in which number for examined objects with a mass of 80 kg in previous MRT examinations.

The deposition values, for example the reference deposition values and the at least one examination deposition value, may each describe a SAR value or a B1 value or a power value, for example a transmission power value. Such a SAR value may, for example, be a whole-body SAR value or a partial-area SAR value. Such a B1 value may be, for example, a B1+RMS value, a B1² value or a B1+² value.

Standards for MRT examinations may stipulate that the averaged specific absorption rate (SAR) must not exceed a specified limit value in a specified time interval in order to prevent damage to the patient due to overheating. This specification may in certain circumstances limit the performance (for example the speed of the MRT examination and/or the number of measured slices and/or the resulting image quality), since compliance with the SAR limits requires the MRT protocol of the MRT examination to be adjusted in order to reduce the power delivered to the patient.

For example, the MRT protocol may define pulse sequences that may be delivered by an MRT system during the MRT examination. The predetermined MRT protocol may have MRT protocol parameters, that may be static or customizable. In order to be able to ensure compliance with at least one specification of deposition values, for example compliance with SAR limit values in the MRT examination, it may be provided that at least one of the MRT protocol parameters of the specified MRT protocol is adjusted or ascertained in a specified MRT protocol optimization procedure. The MRT protocol may be provided to define the MRT examination of the object to be examined by an MRT system. The MRT protocol may include at least one MRT protocol parameter, wherein the MRT protocol parameter may be, for example, a tilt angle to be effected by the MRT apparatus during the MRT examination.

The at least one examination deposition value may be ascertained on the basis of a calibration parameter that is independent of a predetermined MRT protocol of the MRT examination. The ascertained performance value is thus also independent of the specific settings of the MRT protocol, such as, for example, the number of slices or tilt angle.

The calibration parameter may be dependent on the MRT system, for example its performance, and/or the respective examination object, for example the body part of the examination object to be examined. When ascertaining the performance value, the influence of the examination object on a variable derived from the calibration parameter, for example the examination deposition value, is statistically evaluated with the aid of the reference frequency distribution, so that this derived variable is more informative about the MRT system, for example its performance.

The calibration parameter may describe a system control value for generating a calibration radio frequency pulse by the MRT system.

In order to ascertain the calibration parameter, a radio frequency pulse is fed into a transmitter system of the MRT system, for example during the so-called adjustment of a preliminary examination that may be performed before the actual MRT examination. The amplitude of the radio frequency pulse is adjusted (usually: increased) until a calibration field strength B1+, that corresponds to a defined flip angle, may be generated by the MRT system. The radio frequency pulse generated with the adjusted amplitude is then the calibration radio frequency pulse. With the adjusted amplitude as the system control value, the radio frequency pulses to be played out from an MRT protocol that defines a time sequence of the control are scaled. It is possible from this to predetermine the average power to be emitted by the transmitting system. The coil power loss remaining in the transmitting system, for example in a transmitting coil, may be subtracted from this transmitting power in order to determine the active power absorbed in the patient. If this active power is divided by the mass of the object being examined, the SAR value is obtained, for example a whole-body SAR value.

A defined power may be set and the resulting B1+ value may be measured.

In addition to the mass of the examination object, the power—and thus the SAR required for the calibration radio frequency pulse—may depend on other examination parameters, such as the nature of the examination object, a position of an examination area of the examination object and/or an excitation form for exciting the examination object. These examination parameters may not be dependent on a specific MRT protocol.

The method may include receiving at least one examination parameter of the MRT examination, wherein the reference frequency distribution is determined from existing MRT examination data sets of examined reference objects, that each have the at least one examination parameter value and/or whose at least one examination parameter value lies in a range that is assigned to the at least one examination parameter value received.

The at least one examination parameter value of the MRT examination may, for example, be entered by an operator and/or automatically ascertained according to the MRT protocol used and be received by a receiving unit of an evaluation unit for ascertaining the performance value.

A respective one of the MRT examination data sets may include the respective examination deposition value and the respective examination parameter value of the respective MRT examination.

The at least one examination parameter value may be characterized in that it may influence a deposition of the examination object during the MRT examination. The at least one examination parameter value may include a mass of the at least one examination object and/or a position of an examination area of the examination object and/or an excitation form for exciting the examination object and/or an allocation of the object of investigation to a group of persons.

The position of an examination area of the examination object may be characterized, for example, by the part of the body of the examination object to be examined and/or by the examination position of the examination object.

The excitation form for exciting the examination object may be characterized, for example, by whether the excitation is circularly polarized (CP) or elliptically polarized (EP).

The group of persons may be characterized, for example, by the gender of at least one examination object. Another useful distinction would be, for example, competitive athletes versus non-professional athletes, since competitive athletes require a higher transmission power to generate a specific magnetic field due to their higher muscle content.

The reference frequency distribution may be determined from existing MRT examination data sets of multiple MRT reference systems. This increases the database for determining the reference frequency distribution and/or increases the accuracy of the reference frequency distribution. In this case, the multiple MRT reference systems may be the same type in order to increase the informative value of the performance value determined from the reference frequency distribution.

Ascertaining the performance value may include comparing the at least one examination deposition value with the reference frequency distribution and/or a classification of the at least one examination deposition value in the reference frequency distribution.

The reference frequency distribution may be received in the form of a, for example relative, sum frequency (also called cumulative frequency) or a distribution function (cumulative distribution function) and/or derived from a frequency.

The (relative) sum frequency may indicate a cumulative percentage of the reference deposition values up to the examination deposition value. In order to derive the sum frequency from the frequency, the numbers of reference deposition values are to be cumulated, for example added up, according to their sequence in accordance with the frequency. The distribution function may indicate the probability with which a reference deposition value assumes at most the examination deposition value.

Ascertaining the performance value may include ascertaining at least one probability value that describes the probability that a reference object according to the reference frequency distribution has a lower deposition value than the at least one examination deposition value (ascertained with the MRT system), or which describes the probability that a reference object according to the reference frequency distribution has a higher examination deposition value than the at least one examination deposition value (ascertained with the MRT system).

It is possible on the basis of the reference distribution function to determine the probability of the percentage of the (average) population (with the same at least one examination parameter value, such as, for example, weight and/or examination position) that has a smaller deposition value for each MRT examination with the knowledge of the (current) examination deposition value. A better performance value may be derived from a smaller percentage value.

The at least one examination deposition value may include a plurality of examination deposition values. Ascertaining the performance value includes ascertaining a respective probability value for each of the plurality of examination deposition values and ascertaining a mean or median value from the plurality of probability values. The mean value may, for example, be an arithmetic or geometric mean value.

The median value may be the value that lies exactly in the middle of the multiple probability values, that are ordered according to their size. Due to this central position, the median value is often also called the central value. The median value halves the probability values so that half of the probability values lie below and the other half above the median value in the ordered series.

The multiple probability values for ascertaining the mean or median value may be ascertained using examination deposition values that were ascertained in a selected time interval. For example, the probability values are averaged over a time interval of one week or one month. In this way, the influence of outliers in the probability values, that could possibly falsify the performance value, may be better suppressed.

Furthermore, an evaluation unit is provided that is configured to perform a method described above for ascertaining a performance value of an MRT system.

The evaluation unit includes, for example, a receiving unit for receiving a reference frequency distribution of reference deposition values of examined reference objects, a receiving unit for receiving at least one examination deposition value for an MRT examination of at least one examination object by the MRT system, and an ascertaining unit for ascertaining the performance value of the MRT system on the basis of the reference frequency distribution and the at least one examination deposition value. For example, the evaluation unit may include one or more processors and/or memory modules, for example in order to be able to perform arithmetic operations to perform the method. Furthermore, the evaluation unit may, for example, include a user interface with a display unit, for example on at least one monitor, and/or an input unit, for example in order to be able to display and/or evaluate ascertained performance values.

The evaluation unit may, for example, be part of the MRT system whose performance is being ascertained. For example, it may be part of a system control unit of the MRT system. However, it may also be configured separately from the MRT system. For example, it may be connected to the MRT system via a data network. The data network may be a local network, for example. The evaluation unit may also be connected to the MRT system via the Internet. For example, the evaluation unit may be located at a different location, for example a different region and/or a different country, than the MRT system. The MRT system or its performance may be monitored remotely.

A computer program product is also provided that includes a program and is directly loadable into a memory of a programmable evaluation unit and has program code, for example libraries and auxiliary functions, for performing the above described method when the computer program product is executed in the evaluation unit. The computer program product may include software with a source code that still has to be compiled and stored or that only has to be interpreted, or an executable software code that only has to be loaded into the evaluation unit for execution.

The method may be performed rapidly, repeatably identically and in a robust manner by the computer program product. The computer program product may be configured in such a manner that it may perform the method steps by the evaluation unit. The system control unit has respectively the prerequisites, such as for example an appropriate working memory, an appropriate graphics card, and/or an appropriate logic unit so that the respective method steps may be performed efficiently.

The computer program product is, for example stored on a computer-readable medium or stored on a network or server, from where it may be loaded into the processor of a local evaluation unit, that may be directly connected to the MRT system or may be configured as a part of the MRT system. Furthermore, control information of the computer program product may be stored on an electronically readable data carrier. The control information of the electronically readable data carrier may be configured in such a manner that when using the data carrier in the evaluation unit the control information performs the method.

Examples for electronically readable data carriers include a DVD, a magnetic tape, or a USB stick, among others on which electronically readable control information, for example software is stored. When this control information is read from the data carrier and stored in an evaluation unit, the embodiments of the method described above may be performed.

### BRIEF DESCRIPTION OF THE FIGURES

FIG. 1 depicts an evaluation unit with an MRT system and multiple further MRT systems according to an embodiment.

FIG. 2 depicts a method for ascertaining a performance value of an MRT system according to an embodiment.

FIG. 3 depicts a reference frequency distribution of reference deposition values for ascertaining a probability value according to an embodiment.

FIG. 4 depicts a temporal progression of performance values according to an embodiment.

### DETAILED DESCRIPTION

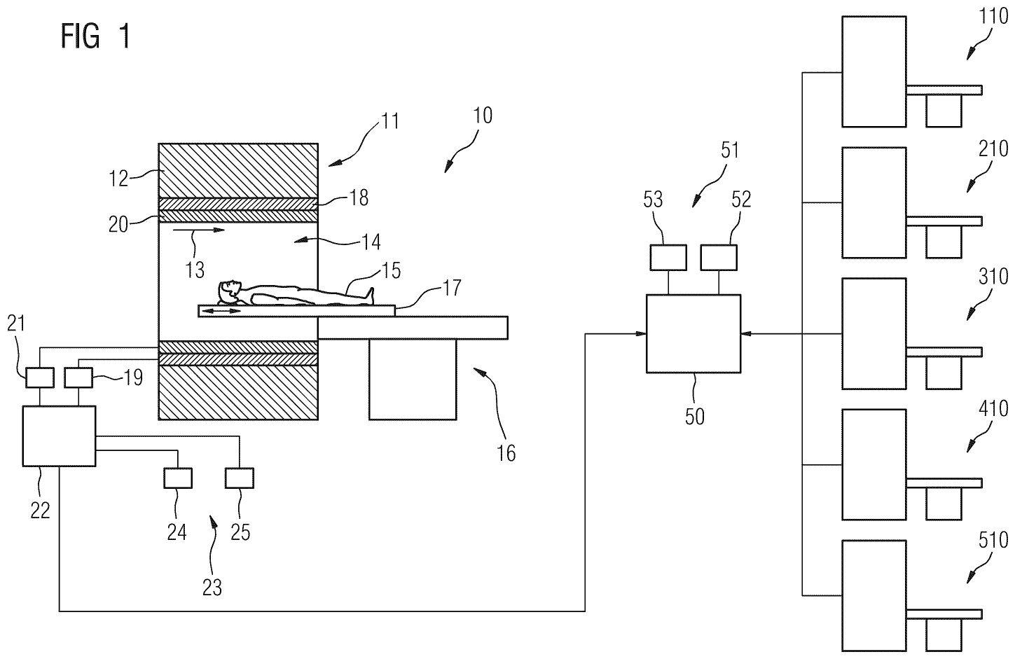

FIG. 1 schematically depicts an MRT system 10. The MRT system 10 includes a magnet unit 11, that includes a main magnet 12 for generating a strong and, for example, temporally constant main magnetic field 13. In addition, the MRT system 10 includes a patient receiving area 14 for receiving a patient 15 as an examination object. The patient receiving area 14 in the current embodiment is cylindrical in shape and is cylindrically surrounded in a peripheral direction by the magnet unit 11. Fundamentally, however, a different design of the patient receiving area 14 may be used. The patient 15 may be pushed by a patient positioning apparatus 16 of the MRT system 10 into the patient receiving area 14. The patient positioning apparatus 16 includes a patient table 17 that is configured so as to be able to move within the patient receiving area 14. The field of view of the MRT system may be located in the center of the patient receiving area 14. Therefore, a position of an examination area of the patient 15 changes depending on the position in which the patient table 17 is moved with the patient 15.

Furthermore, the magnet unit 11 has a gradient coil unit 18 for generating magnetic field gradients used for location encoding during imaging. The gradient coil unit 18 is controlled by a gradient control unit 19 of the MRT system 10. The magnet unit 11 further includes a radio frequency antenna unit 20, which in the present embodiment is configured as a body coil permanently integrated into the MRT system 10. The radio frequency antenna unit 20 is controlled by a radio frequency antenna control unit 21 of the MRT system 10 and radiates radio frequency pulses into an examination space that is essentially formed by a patient receiving area 14 of the magnetic resonance apparatus 10, i.e. the radio frequency antenna unit 20 and the radio frequency antenna control unit 21 form its transmission system of the MRT system here. The main magnetic field 13 generated by the main magnet 12 excites atomic nuclei by irradiating the radio frequency pulses. Magnetic resonance signals are generated by relaxing the excited atomic nuclei. The radio frequency antenna unit 20 is designed to receive the magnetic resonance signals.

The MRT system 10 has a system control unit 22 for controlling the main magnet 12, the gradient control unit 19, and for controlling the radio frequency antenna control unit 21. The system control unit 22 centrally controls the MRT system 10, such as, for example, the execution of a predetermined MRT protocol. In addition, the system control unit 22 includes a reconstructing unit, not shown in detail, for reconstructing magnetic resonance signals acquired during the magnetic resonance examination. Furthermore, the MRT system 10 includes a user interface 23 that is connected to the system control unit 22. Control information, such as for example imaging parameters, and also reconstructed magnetic resonance images may be displayed on a display unit 24, for example on at least one monitor of the user interface 23 for a medical operator. Furthermore, the user interface 23 includes an input unit 25, by which information and/or parameters may be input by the medical operator during a measuring procedure.

The system control unit 22 of the MRT system 10 is connected to an evaluation unit 50. The system control unit 22 transmits to the evaluation unit 50 at least one examination deposition value and/or information for determining at least one examination deposition value.

Furthermore, the evaluation unit 50 is connected to multiple further MRT systems 110, 210, 310, 410, 510. These further MRT systems 110, 210, 310, 410, 510 transmit information to the evaluation unit 50 for determining a reference frequency distribution of reference deposition values of patients who were examined with these further MRT systems 110, 210, 310, 410, 510. The other systems 110, 210, 310, 410, 510 may therefore also be regarded as reference MRT systems.

Furthermore, the evaluation unit 50 includes a user interface 51 with a display unit 52, for example a monitor, and an input unit 53, by which information, for example performance values of the MRT system 10, may be displayed to an operator and/or information may be entered by an operator.

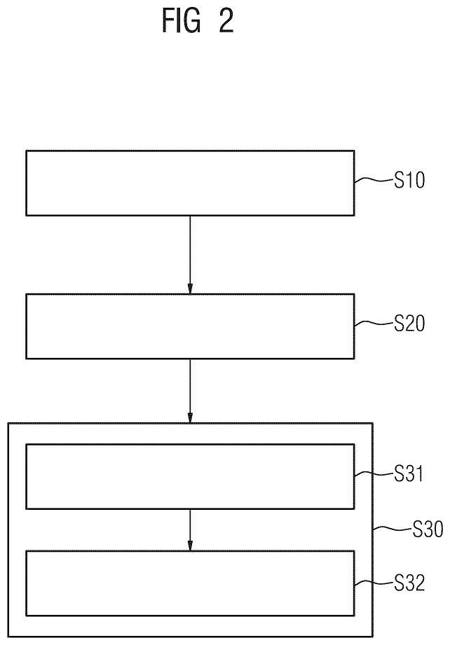

The evaluation unit 50 includes at least one processor and/or at least one memory module, in order to be able to perform a computer-implemented method for ascertaining a performance value of an MRT system 10. Such a method is schematically depicted in FIG. 2.
In S10, a reference frequency distribution of reference deposition values of examined reference objects is received. The reference objects are for example patients who have been examined with reference MRT systems, for example the multiple other MRT systems 110, 210, 310, 410, 510. The multiple other MRT systems may not be just five MRT systems (as shown), but an entire fleet of MRT systems.
In S20, at least one examination deposition value for an MRT examination of at least one examination object, for example the patient 15, is ascertained and/or received by the MRT system 10.
In S30, a performance value of the MRT system is ascertained using the reference frequency distribution and the at least one deposition value. For example, in order to ascertain the performance value in S31, multiple probability values may be ascertained, that are averaged in S32.

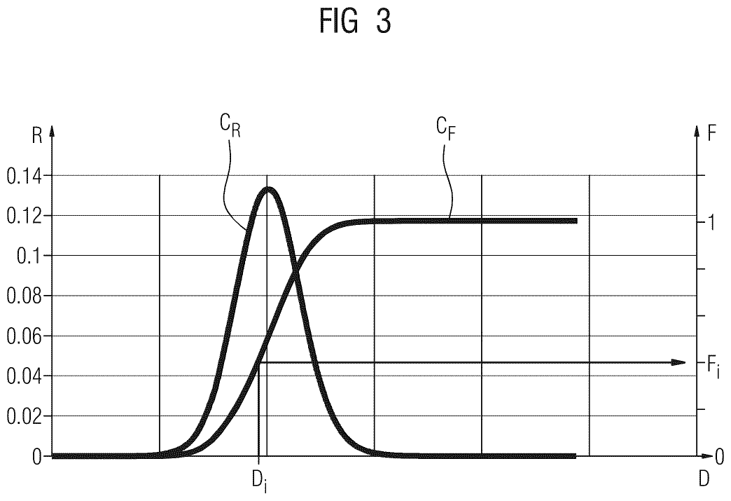

FIG. 3 explains the method in more detail. A reference frequency distribution of reference deposition values of examined reference objects is shown here in the form of a relative frequency C_{R} and a relative sum frequency C_{F}. The relative frequency C_{R} may usually be derived from an absolute frequency. The left vertical axis R shows the value R of the relative frequency C_{R}. The right vertical axis R shows the value F of the relative sum frequency C_{F}, that lies between 0 and 1 or 100 percent. The frequency may be in the form of a Gaussian distribution, as in the case shown.

The horizontal axis D indicates a deposition value. The deposition value may be a SAR value, for example, but other types of deposition values are also conceivable. The SAR value, for example, may be calculated as follows: D=P/m where P is the power absorbed by the patient that is required to generate a calibration radio frequency pulse by the transmitter system of the MRT system 10. The absorbed power P determined by such a calibration may therefore be regarded as a calibration parameter.

Since the calibration radio frequency pulse may be a radio frequency pulse that is independent of an MRT protocol to be performed during the MRT examination, the resulting absorbed power in the patient is also independent of this. This value may be determined, for example, in a preliminary examination to be performed before the actual MRT examination. m is the mass of the patient 15, i.e. D is standardized to the mass of the patient 15 using this value.

For safety reasons, the SAR value may be ascertained anyway for performing an MRT examination in order not to exceed any SAR limits. This means that the deposition value D may be available without further effort, that is why the performance value may be ascertained particularly efficiently.

If, for example, the examination deposition value Dᵢ is ascertained as the SAR value, the probability value Fᵢ=C_{F}(Dᵢ) may be determined from the relative sum frequency C_{F}. This probability value Fᵢ describes the probability that a reference object according to the reference frequency distribution has a lower SAR value than the examination deposition value Dᵢ. Consequently, 1−Fᵢ describes the probability that a reference object according to the reference frequency distribution has a higher SAR value than the examination deposition value Dᵢ. A probability value of 0.5 would mean that the MRT system 10 shows an average behavior, for example performance, compared to the reference MRT systems. In the example shown, the probability value Fᵢ is approximately 0.4.

Various examination parameter-specific reference frequency distributions may be provided. A suitable reference frequency distribution is selected or received by the evaluation unit 50 from the reference frequency distribution according to at least one examination parameter value of the MRT examination by the MRT system 10. Suitable examination parameter values may be the mass of patient 15 and/or a position of an examination area of patient 15 and/or an excitation form for exciting the patient 15 and/or an allocation of patient 15 to a group of persons. For example, the reference frequency distribution may be specific for 60-65 kg women whose heads are examined with a CP excitation. The position of an examination area of the patient 15 may also be defined in the form of a position of the patient table 17 along its travel path that may correspond to a part of the body of the patient 15.

The reference frequency distribution used for ascertaining the performance value may be ascertained from existing MRT examination data sets of examined reference objects that each have the at least one examination parameter value and/or whose at least one examination parameter value lies in a range that is assigned to the at least one examination parameter value received. For example, a corresponding mass range may have a width of 5 kg or a range of a position of the patient table may have a width of 10-15%.

The MRT examination data sets may be generated, for example, by the further MRT systems 110, 210, 310, 410, 510. The reference frequency distribution is based on as many previously performed MRT examinations as possible. The other MRT systems 110, 210, 310, 410, 510 may be the same type as the MRT system 10.

All available reference frequency distributions may be stored in a central database, from which a suitable reference frequency distribution for ascertaining the performance value may be retrieved and, for example, may be received by the evaluation unit 50. The database may also be part of the evaluation unit 50.

Multiple, for example all, MRT systems (for example therefore also the MRT system 10 in addition to the other MRT systems 110, 210, 310, 410, 510) may provide information, for example ascertained deposition values, for generating the reference frequency distributions, such providing MRT systems may also be referred to as MRT reference systems.

It is possible for a performance value to be determined solely from a single determined probability value Fᵢ. In one case, the performance value is identical to the ascertained probability value Fᵢ, i.e. P=Fᵢ.

However, it is also possible for multiple MRT examinations to be performed on the MRT system 10, in each of which an examination deposition value, for example a SAR value, is ascertained. This means that there are multiple examination deposition values. A probability value Fₓ may then be determined for each of the multiple examination deposition values. For example, a mean value or median value may be ascertained from these probability values Fₓ.

For example, an arithmetic mean value may be formed in accordance with:

$${\overline{F}}_{arithm} = \frac{1}{n} {\sum }_{x = 1}^{n} {F}_{x}$$

or, for example, a geometric mean value may be formed in accordance with:

$${\overline{F}}_{geom} = \sqrt[n]{{\prod }_{x = 1}^{n} {F}_{x}}$$

For example, the averaging may be performed using probability values Fₓ derived from MRT examinations that took place in a specific time interval. For example, a performance value may be determined on a weekly or monthly basis.

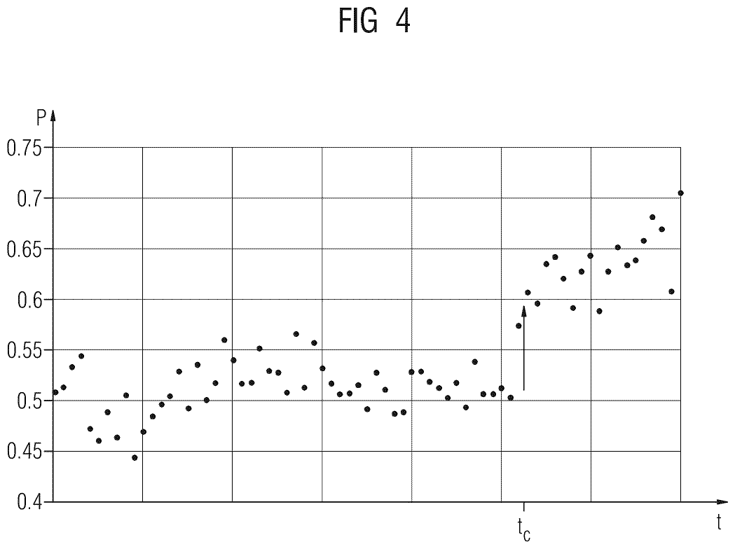

FIG. 4 depicts a possible temporal progression of the performance value of an MRT system 10. Each point corresponds to a performance value Pₓ at a specific time t, for example. It may be seen from the diagram that the performance value P has increased significantly from the point in time t_{c}. This indicates that a change may have taken place in the MRT system 10. This change may, for example, have been caused by a deterioration of a component of the MRT system 10. For example, according to the example shown, a higher performance value may correspond to a higher amplitude required to generate a calibration radio frequency pulse by the MRT system 10. By ascertaining the performance value, in which statistical scatter is taken into account with the aid of the reference frequency distribution, possible problems may be detected early and reliably and eliminated proactively, such as defective or aged transmitting elements. Possible system failures may thus be prevented. However, the influence of any software updates may also be observed with the help of the ascertained performance values.

The method for ascertaining the performance values may be used to distinguish between different possible influences for a specific behavior of an MRT system, such as, for example, the influence of an MRT protocol used, an examined patient or the MRT system itself. The influence of the individual patient may be reduced, ideally eliminated, with the help of the performance value in that a statistical analysis of multiple patients is carried out. Finally, it is likely that the average characteristics of the patients are the same and do not depend on the specific MRT system with which they are examined.

The methods described in detail above and the evaluation unit and MRT system shown are merely examples of embodiments that may be modified by the skilled person in various ways without departing from the scope of the invention. Furthermore, the use of the indefinite articles “a” or “one” does not exclude the possibility that the features in question may be present more than once. Similarly, the term “unit” does not exclude the possibility that the components in question include multiple interacting sub-components, that may also be spatially distributed.

It is to be understood that the elements and features recited in the appended claims may be combined in different ways to produce new claims that likewise fall within the scope of the present embodiments. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present embodiments have been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A computer-implemented method for ascertaining a performance value of an MRT system, the method comprising:
receiving a reference frequency distribution of reference deposition values of examined reference objects;
receiving at least one examination deposition value, for an MRT examination of at least one examination object by the MRT system; and
ascertaining the performance value of the MRT system using the reference frequency distribution and the at least one deposition value.

2. The method of claim 1, wherein each of the reference deposition values and the at least one examination deposition value each describe at least one of a SAR value, a B1 value, or a power value.

3. The method of claim 2, wherein the power value comprises a transmission power value.

4. The method of claim 1, wherein the at least one examination deposition value is ascertained based on a calibration parameter that is independent of a predetermined MRT protocol of the MRT examination.

5. The method of claim 4, wherein the calibration parameter describes a system control value for generating a calibration radio frequency pulse by the MRT system.

6. The method of claim 1, wherein the reference frequency distribution is determined from reference deposition values and/or existing MRT examination data sets of multiple MRT reference systems.

7. The method of claim 6, wherein the multiple MRT reference systems are of a same type.

8. The method of claim 6, further comprising:
receiving at least one examination parameter value of the MRT examination data sets of examined reference objects that each have the at least one examination parameter value and/or whose at least one examination parameter value lies in a range that is assigned to the at least one examination parameter value received.

9. The method of claim 8, wherein the at least one examination parameter value comprises at least one of a mass of the at least one examination object, a position of an examination area of the examination object, an excitation form for exciting the examination object, or an allocation of the examination object to a group of persons.

10. The method of claim 1, wherein the reference frequency distribution is received as a cumulative frequency or a cumulative distribution function and/or is derived from a frequency.

11. The method of claim 1, wherein ascertaining the performance value includes ascertaining at least one probability value that describes a probability that a reference object according to the reference frequency distribution has a lower deposition value than the at least one examination deposition value, or that describes a probability that a reference object according to the reference frequency distribution has a higher examination deposition value than the at least one examination deposition value.

12. The method of claim 1, wherein the at least one examination deposition value comprises multiple examination deposition values, wherein ascertaining the performance value includes:
ascertaining a respective probability value for each of the multiple examination deposition values; and
ascertaining a mean value or median value from the ascertained probability values.

13. The method of claim 12, wherein the ascertained probability values for ascertaining a mean value or a median value are ascertained using examination deposition values that were ascertained in a selected time interval.

14. An evaluation unit comprising:
a receiving unit configured to receive a reference frequency distribution of reference deposition values of examined reference objects;
a receiving unit configured to receive at least one examination deposition value for an MRT examination of at least one examination object by a MRT system; and
an ascertaining unit configured to ascertain a performance value of the MRT system based on the reference frequency distribution and the at least one examination deposition value.

15. The evaluation unit of claim 14, further comprising:
a database and multiple MRT reference systems that provide reference deposition values to the database for generating at least one reference frequency distribution.

16. A non-transitory computer implemented storage medium that stores machine-readable instructions for ascertaining a performance value of an MRT system, the machine-readable instructions executable by at least one processor, the machine-readable instructions comprising:
receiving a reference frequency distribution of reference deposition values of examined reference objects;
receiving at least one examination deposition value, for an MRT examination of at least one examination object by the MRT system; and
ascertaining the performance value of the MRT system using the reference frequency distribution and the at least one deposition value.

17. The non-transitory computer implemented storage medium of claim 16, wherein each of the reference deposition values and the at least one examination deposition value each describe at least one of a SAR value, a B1 value, or a power value.

18. The non-transitory computer implemented storage medium of claim 16, wherein the at least one examination deposition value is ascertained based on a calibration parameter that is independent of a predetermined MRT protocol of the MRT examination.

19. The non-transitory computer implemented storage medium of claim 18, wherein the calibration parameter describes a system control value for generating a calibration radio frequency pulse by the MRT system.

20. The non-transitory computer implemented storage medium of claim 16, wherein the reference frequency distribution is determined from reference deposition values and/or existing MRT examination data sets of multiple MRT reference systems.
